(19) United States
(12) Patent Application Publication
(10) Pub. No.: US 2021/0031030 A1
(43) Pub. Date: Feb. 4, 2021

(54) COSMETIC DEVICE

(71) Applicant: LG HOUSEHOLD & HEALTH CARE LTD., Seoul (KR)

(72) Inventors: Seon Ung MIN, Seoul (KR); Jin Won PARK, Seoul (KR)

(73) Assignee: LG HOUSEHOLD & HEALTH CARE LTD., Seoul (KR)

(21) Appl. No.: 16/893,059

(22) Filed: Jun. 4, 2020

(30) Foreign Application Priority Data
Aug. 2, 2019 (KR) 10-2019-0094475

(51) Int. Cl. A61N 1/26; A61H 15/00; A61N 1/08; A61N 1/04; H05B 45/20

(52) U.S. Cl. CPC A61N 1/26; A61H 15/0092; A61N 1/08; A61N 1/0408; A61H 2201/1215; H05B 45/20; A61H 2015/0071; A61H 2201/10; A61H 2015/0042; A61N 1/0472

(57) ABSTRACT

Disclosed herein is a cosmetic device. The cosmetic device includes a pair of shafts secured to a handle, a pair of rotating bodies rotatably coupled to the pair of shafts, and a pair of vibration motors configured to transmit vibration to the pair of rotating bodies. The vibration motors are installed in the shafts, respectively.

### CROSS-REFERENCE TO RELATED APPLICATION

The present application claims priority under 35 U.S.C. § 119(a) to Korean patent application number 10-2019-0094475, filed on Aug. 2, 2019, in the Korean Intellectual Property Office, the entire disclosure of which is incorporated herein by reference.

### BACKGROUND

### 1. Technical Field

The present disclosure relates to a cosmetic device. More particularly, the present disclosure relates to a cosmetic device configured such that a vibration motor is built in a shaft portion serving as a rotating shaft of a rotating body, so that the vibration of the vibration motor can be directly transmitted to the rotating body, and vibration efficiency can be increased.

### 2. Related Art

Generally, a cosmetic device designed to massage the skin of the face, the arm or the like using a spherical ball includes, as main components, a handle, and a pair of rotating bodies (rollers) provided on an end of the handle. If a user holds the handle with his or her hand and moves the cosmetic device while pressing the rotating body against the skin, the skin is caught between the rotating bodies, so that pores are contracted or opened and thus the dirt of the pores are removed.

The cosmetic device is generally configured such that the pair of rotating bodies is rotatably mounted on the handle, only an end of each rotating body has a hole, and the rotating body is supported on a shaft that is a support shaft via a bearing member.

In addition to these general components, a cosmetic device with a vibration motor for vibrating the rotating body to help skin tissue to be relaxed or contracted and thereby allow active ingredients in cosmetics to be effectively absorbed has been proposed and used.

However, the conventional cosmetic device with the vibration motor is problematic in that the vibration motor is provided not in the rotating body but in a handle gripped by the hand, so that the vibration of the vibration motor is not directly transmitted to the rotating body but is distributed to the handle, and consequently the vibration efficiency of the rotating body is reduced.

### SUMMARY

The present disclosure has been made to solve the above-mentioned problems and provides a cosmetic device configured to allow vibration of a vibration motor to be directly transmitted to a rotating body.

### Technical Solution

According to an aspect of the present disclosure, there may be provided a cosmetic device, including a pair of shafts secured to a handle; a pair of rotating bodies rotatably coupled to the pair of shafts; and a pair of vibration motors configured to transmit vibration to the pair of rotating bodies, wherein the vibration motors may be installed in the shafts, respectively.

The shafts may be independently provided on opposite sides of a partition wall that is formed to have a predetermined size along a center line inside a front end of the handle, so that the shafts may be electrically separated from each other.

Each of the shafts may include a cylindrical shaft member inserted into a hole of each of the rotating bodies; and a fixing member extending from the shaft member in a shape of a plate and fixedly coupled to the handle.

The shaft member may include an installation space in which each of the vibration motors is inserted and installed.

The cosmetic device may further include a battery installed in the handle to supply power; and a circuit board installed in the handle and configured to perform a vibration massage function by the vibration motor and a microcurrent massage function by a microcurrent module.

The microcurrent module may be configured such that it causes “positive” current of the battery to flow through any one of the shafts and a bearing to any one of the rotating bodies, thus allowing this rotating body to become the “positive” electrode, and it causes “negative” current of the battery to flow through a remaining one of the shafts and the bearing to the remaining one of the rotating bodies, thus allowing this rotating body to become the “negative” electrode.

The microcurrent module may form a closed circuit around the battery if the rotating body that is the “positive” electrode and the rotating body that is the “negative” electrode simultaneously come into contact with the skin, thus transmitting the microcurrent to the skin.

According to an aspect of the present disclosure, there may be provided a cosmetic device, including a main control unit; a mode selector transmitting mode signal information selected by manipulating a button to the main control unit; an LED generator giving an alarm through an LED lamp to indicate what a current mode is, in response to the mode signal information transmitted from the main control unit; a motor driver driving a vibration motor installed inn a shaft that may rotate a rotating body, in response to the mode signal information transmitted from the main control unit; and a microcurrent generator generating microcurrent to cause the microcurrent to flow in the rotating body, in response to the mode signal information transmitted from the main control unit.

The mode selector may transmit soft mode signal information selected by pressing the button once, and power mode signal information selected by pressing the button twice to the main control unit.

The main control unit may perceive a charge or discharge state of the battery to provide information about the charge/discharge state to the LED generator, and the LED generator may cause the LED lamp to emit green light if the soft mode signal information is transmitted from the main control unit, and may cause the LED lamp to emit blue light if the power mode signal information is transmitted from the main control unit, and may cause the LED lamp to emit red light if information about the discharge state of the battery is transmitted from the main control unit.

The motor driver may cause the vibration motor to be driven at a level of 60% to 80% of rating, if the soft mode signal information is transmitted from the main control unit, and may cause the vibration motor to be driven at a rating level, if the power mode signal information is transmitted from the main control unit.

The microcurrent generator may generate current of 25 uA to 35 uA if the soft mode signal information is transmitted from the main control unit, and may generate current of 90 uA to 110 uA if the power mode signal information is transmitted from the main control unit.

A cosmetic device according to the present disclosure is advantageous in that a vibration motor is installed in a shaft member of a shaft inserted into a rotating body, so that the vibration of the vibration motor can be directly transmitted to the rotating body, and thereby vibration efficiency can be increased, and in that the vibration is not transmitted to the hand through a handle, so that a user's satisfaction as well as a product quality can be improved.

Furthermore, a cosmetic device according to the present disclosure is advantageous in that a vibration motor is provided not on a handle side but on a rotating-body side, so that a spare space is provided in the handle side unlike the related art in which the vibration motor is provided on the handle side, and consequently it is possible to make better use of a space and to achieve miniaturization using the spare space.

Furthermore, a cosmetic device according to the present disclosure is advantageous in that a fixing member is formed to extend from a shaft member of a shaft in the shape of a plate, so that the fixing member may be easily fixed to the handle using a screw, thus simplifying a component for fixing the shaft, and in that an electric wire is connected to the screw, so that microcurrent flows through the shaft to a rotating body, thus realizing a microcurrent massage function.

### BRIEF DESCRIPTION OF THE DRAWINGS

FIG. 1 is an entire plan view of a cosmetic device in accordance with an embodiment of the present disclosure.

FIG. 2 is an enlarged view illustrating an internal portion of the cosmetic device in accordance with the embodiment of the present disclosure.

FIG. 3 is a partial sectional view of the cosmetic device in accordance with the embodiment of the present disclosure.

FIG. 4 is a partial exploded view of the cosmetic device in accordance with the embodiment of the present disclosure.

FIG. 5 is a block diagram illustrating an operation of the cosmetic device in accordance with the embodiment of the present disclosure.

### DETAILED DESCRIPTION

The objectives of the present disclosure, specific advantages, and novel characteristics will become more apparent from the following description and preferred embodiments with reference to the accompanying drawings. It should be noted that the same reference numerals shown in the drawings will denote the same components. Furthermore, in the description of the present disclosure, if it is determined that the detailed description of the related art unnecessarily makes the gist of the present disclosure obscure, its detailed description will be omitted.

Hereinafter, a preferred embodiment of the present disclosure will be described in detail with reference to the accompanying drawings.

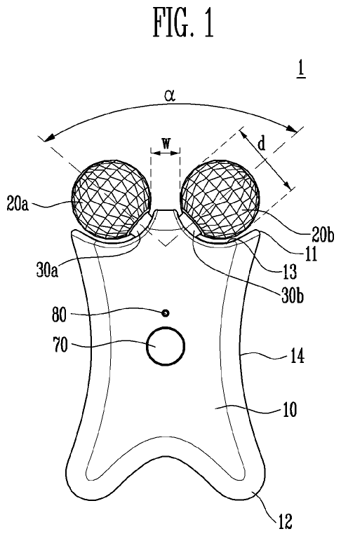

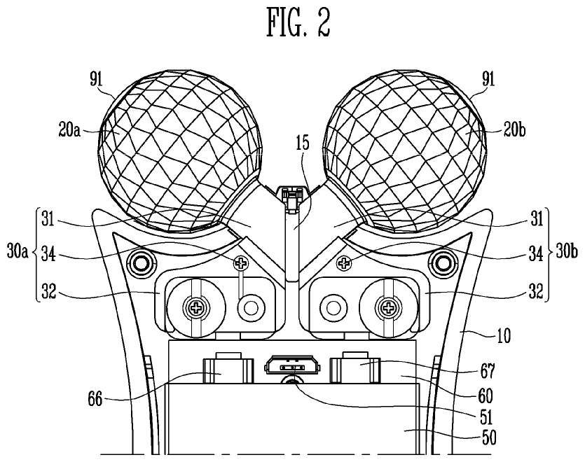

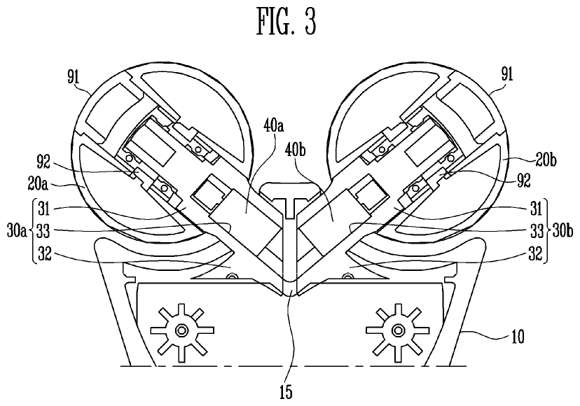

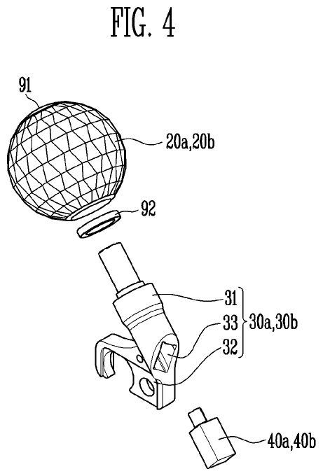

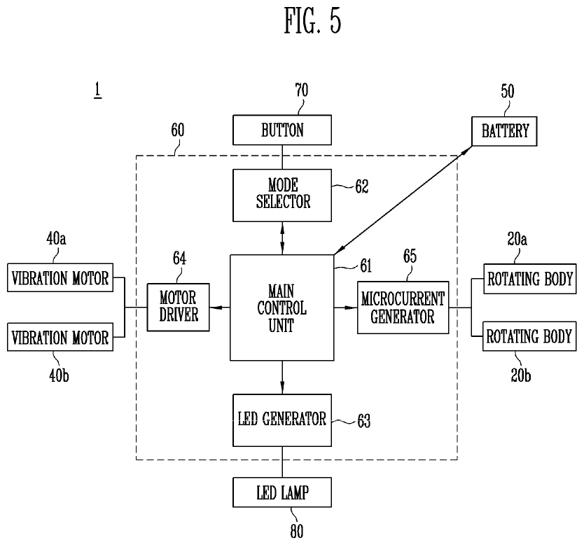

FIG. 1 is an entire plan view of a cosmetic device in accordance with an embodiment of the present disclosure, FIG. 2 is an enlarged view illustrating an internal portion of the cosmetic device in accordance with the embodiment of the present disclosure, FIG. 3 is a partial sectional view of the cosmetic device in accordance with the embodiment of the present disclosure, FIG. 4 is a partial exploded view of the cosmetic device in accordance with the embodiment of the present disclosure, and FIG. 5 is a block diagram illustrating an operation of the cosmetic device in accordance with the embodiment of the present disclosure.

Referring to FIGS. 1 to 5, a cosmetic device 1 in accordance with an embodiment of the present disclosure may perform a compression massage function, a meridian scraping massage function, a vibration massage function, and a microcurrent massage function, and includes a handle 10, rotating bodies 20a and 20b, shafts 30a and 30b, vibration motors 40a and 40b, a battery 50, a circuit board 60, a button 70, and an LED lamp 80.

The handle 10 has a predetermined length and thickness to allow a user to easily grip it, and is configured such that a pair of rotating bodies 20a and 20b is rotatably coupled to a front end thereof. The pair of rotating bodies 20a and 20b may be coupled to branch at a predetermined angle from a center line of the handle 10. If the cosmetic device reciprocates along the center line of the handle 10 in a state where the user grips the handle 10 and makes the rotating bodies 20a and 20b come into contact with a body region, tightening and releasing are repeatedly performed when the skin is pulled by the rotating bodies 20a and 20b.

The handle 10 includes a pair of front protrusions 11 protruding from both sides of the front end thereof, and a pair of rear protrusions 12 protruding from both sides of a rear end that is opposite to the front end. The rotating bodies 20a and 20b may be located on the front end of the handle 10, and the front protrusions 11 may be located on left and right sides of the pair of rotating bodies 20a and 20b, respectively. The pair of rear protrusions 12 may diverge from the rear end of the handle 10 at an oblique angle. The meridian-scraping massage function may be performed using the rear protrusions 12 on the rear end of the cosmetic device 1.

An inside portion 13 of the front protrusion 11 adjacent to each of the rotating bodies 20a and 20b may have a shape corresponding to that of each of the rotating bodies 20a and 20b. For example, if each of the rotating bodies 20a and 20b has a spherical shape, the inside portion 13 of the front protrusion 11 may have a hemispherical shape when seen from a plane. Furthermore, the inside portion 13 of the front protrusion 11 may be spaced apart from each of the rotating bodies 20a and 20b by a predetermined distance so as not to interfere with the rotation of each of the rotating bodies 20a and 20b.

The front protrusion 11 is shaped to protrude upwards while surrounding at least a portion of each of the rotating bodies 20a and 20b. The protruding height of the front protrusion 11 may be set so as not to interfere with a rotating shaft of each of the rotating bodies 20a and 20b. To be more specific, the protruding height may be set so as not to cover a cap 91 located on the rotating shaft of each of the rotating bodies 20a and 20b. This is intended to easily perform an assembly process of coupling the cap 91 to each of the rotating bodies 20a and 20b.

Furthermore, the front protrusion 11 may perform the meridian scraping massage function as in the rear protrusion 12. However, the protruding height of the front protrusion 11 may be set as low as possible so that an outside portion of each of the rotating bodies 20a and 20b is exposed as much as possible. This allows a region in which it is inconvenient to simultaneously employ the pair of rotating bodies 20a and 20b when the vibration massage function is performed to be easily massaged with only any one of the rotating bodies 20a and 20b.

The front protrusions 11 and the rear protrusions 12 may be integrated with the handle 10, and may be made of a rigid body. Alternatively, a surface of each front protrusion 11 that is in contact with the skin or a surface of each rear protrusion 12 that is in contact with the skin may be covered with an elastic body such as rubber, thus preventing excessive pressure from being applied to the skin.

When viewed from a side, the handle 10 may be gently curved. This improves grip feeling and makes it easier to grip the cosmetic device with a user's hand, as compared to the straight-shaped handle 10.

When viewed from a plane, the outside portion 14 of the handle 10 may be curved. A user can effectively massage a wide body region such as the side, the back, or the thigh, using the outside portion 14 of the handle 10.

Some or all of material of the handle 10 may be selected from any one of synthetic resin, metal, or combinations thereof. The present disclosure is not limited thereto.

The circuit board 60 and the battery 50 may be installed in the handle 10, while the button 70 and the LED lamp 80 may be provided on an outer surface of the handle 10. Furthermore, a partition wall 15 of a predetermined size may be formed inside the front end of the handle 10 along the center line. This partition wall 15 serves to electrically separate the pair of shafts 30a and 30b that contribute to the microcurrent massage function.

The rotating bodies 20a and 20b are rotatably coupled to the shafts 30a and 30b serving as the rotating shaft on the front end of the handle 10. If a user moves the handle 10 with the rotating bodies 20a and 20b being in contact with the skin, the compression massage function for massaging the skin is performed while the rotating bodies 20a and 20b are rolled.

Furthermore, in the case of performing the vibration massage function, vibration may be transmitted from the vibration motors 40a and 40b to the rotating bodies 20a and 20b to vibrate the rotating bodies.

Furthermore, in the case of performing the microcurrent massage function, one rotating body 20a may serve as a “positive” electrode, while the other rotating body 20b may serve as a “negative” electrode. If both the rotating body 20a of the “positive” electrode and the rotating body 20b of the “negative” electrode simultaneously come into contact with the skin, a closed circuit is formed around the battery 50, so that the microcurrent is transmitted to the skin. In this embodiment, it is illustrated that the pair of rotating bodies 20a and 20b and the skin form the closed circuit around the battery 50 to transmit the microcurrent to the skin. However, without being limited thereto, when an electrode (not illustrated) is provided on the handle 10 and a user holds the handle 10, the user comes into contact with the electrode, so that at least one of the rotating bodies 20a and 20b, the hand, and the skin form the closed circuit around the battery 50 and thereby the microcurrent can be transmitted to the skin. In this case, the microcurrent massage function may be performed by only any one of the pair of rotating bodies 20a and 20b.

The rotating bodies 20a and 20b may be positioned between the pair of front protrusions 11, and the rotating shafts of the respective rotating bodies 20a and 20b may be branched from the center line of the handle 10 in a Y shape. An angle between the rotating shaft of one rotating body 20a and the rotating shaft of the other rotating body 20b is referred to as an opening angle α between the rotating bodies 20a and 20b. Preferably, the opening angle α between the rotating bodies 20a and 20b is from 90 degrees to 110 degrees. This can be effective to massage a face region, such as the cheek, the side of the mouth, or the eye. In this embodiment, the opening angle α between the rotating bodies 20a and 20b is 99 degrees.

Furthermore, the shortest distance w between outer circumferences of the pair of rotating bodies 20a and 20b preferably ranges from 8 mm to 9 mm. The diameter d of each of the pair of rotating bodies 20a and 20b preferably ranges from 21 mm to 27 mm. When the shortest distance w between the rotating bodies 20a and 20b and the diameter d of each of the pair of rotating bodies 20a and 20b have the above-described ranges, the effect of picking up the skin of a narrow portion such as the eye or the side of the mouth is appropriately exhibited, and thus massage effect can be further improved. In this embodiment, the shortest distance w between the rotating bodies 20a and 20b is 8.75 mm, and the diameter d of each of the rotating bodies 20a and 20b is 24 mm.

Each of the rotating bodies 20a and 20b may have a spherical shape, an elliptical spherical shape, or combinations thereof. Each of the rotating bodies 20a and 20b may have on a surface thereof an uneven portion or a projection of various shapes. Each of the rotating bodies 20a and 20b may be formed of silicone, metal, or plastic material having proper hardness. However, since one rotating body 20a serves as the “positive” electrode and the other rotating body 20b serves as the “negative” electrode so as to perform the microcurrent massage function, each of the rotating bodies 20a and 20b is preferably formed of metal material having conductivity.

As described above, in this embodiment, two rotating bodies 20a and 20b are illustrated. However, three or four rotating bodies may be provided. If the compression massage function for tightening the skin, the vibration massage function, or the microcurrent massage function is performed between the rotating bodies 20a and 20b while the rotating bodies 20a and 20b are independently rotated, the number or arrangement of the rotating bodies 20a and 20b is not particularly limited.

The shafts 30a and 30b may be secured to the handle 10 so that the rotating bodies 20a and 20b are rotatably coupled to the handle 10. One pair of shafts that are as many as the rotating bodies 20a and 20b may be provided.

As will be described later, since the pair of shafts 30a and 30b serves as one component for performing the microcurrent massage function, the shafts are formed of metal material having excellent conductivity. In order to electrically separate the shafts from each other, they are independently provided on opposite sides of the partition wall 15 that is formed to have a predetermined size along the center line inside the front end of the handle 10.

The pair of shafts 30a and 30b is installed on the front end of the handle 10 to flare outwards with respect to the center line. Each shaft may include a cylindrical shaft member 31 that is coupled to the rotating body 20a or 20b, and a fixing member 32 that extends from the shaft member 31 in the form of a plate and is coupled to the handle 10.

The shaft member 31 of the shaft 30a or 30b is inserted into a hole (not illustrated) of the rotating body 20a or 20b. The shaft member 31 may be fitted into a bearing 92 provided on an inner circumference of the hole of the rotating body 20a or 20b. Thus, the rotating body 20a or 20b is configured to be independently rotated while being coupled to the shaft member 31 of the shaft 30a or 30b.

An installation space 33 may be provided in the shaft member 31 inserted into the hole of the rotating body 20a or 20b, so that the vibration motor 40a or 40b is inserted into the installation space. The installation space 33 may be provided to correspond to the size and shape of the vibration motor 40a or 40b. Thus, the vibration motor 40a or 40b is driven by a motor driver 64 of the circuit board 60 while being accommodated in the shaft member 31 of the shaft 30a or 30b.

The fixing member 32 of the shaft 30a or 30b may be fixedly coupled to the handle 10. The fixing member 32 may be fastened to the handle 10 by a screw 34.

The screw 34 does not only serve to fasten the fixing member 32 but also serves to connect an electric wire (not illustrated), thus allowing the microcurrent to flow through the shaft 30a or 30b to the rotating body 20a or 20b. Furthermore, the circuit board 60 is configured so that an end of the fixing member is operated in conjunction with a microcurrent generator 65 of the circuit board 60 when the screw 34 is fastened to the fixing member 32, thus allowing the microcurrent to flow through the screw 34 to the shaft 30a or 30b without the electric wire.

The vibration motor 40a or 40b may be inserted into the installation space 33 formed in the shaft member 31 of the shaft 30a or 30b, and may be driven by the motor driver 64 of the circuit board 60 while being accommodated in the shaft member 31 of the shaft 30a or 30b. Thus, the vibration motor 40a or 40b may directly transmit vibration to the rotating body 20a or 20b.

Of course, the vibration motor 40a or 40b may be installed in only any one of the shaft members 31 of the shafts 30a and 30b inserted into the holes of the rotating bodies 20a and 20b.

The vibration motor 40a or 40b may be driven in a soft mode and a power mode depending on the selection of a mode selector 62 of the circuit board 60. In the case of selecting the soft mode, the vibration motor 40a or 40b may be driven at 10,500 RPM that corresponds to about 70% of rating. In the case of selecting the power mode, the vibration motor 40a or 40b may be driven at 15,000 RPM that corresponds to the rating level.

The battery 60 may be installed in the handle 10. The battery 50 may be charged through an USB port 51. The charged power may be supplied through the circuit board 60 to a component where power is needed, such as the vibration motors 40a and 40b or the LED lamp 80.

The circuit board 60 may be installed in the handle 10. As the button 70 is pushed, the circuit board may be activated (ON state of the cosmetic device 1) or deactivated (OFF state of the cosmetic device 1). The circuit board 60 may include a main control unit 61, a mode selector 62, an LED generator 63, a motor driver 64, and a microcurrent generator 65 so that the cosmetic device 1 performs the vibration massage function and the microcurrent massage function. Furthermore, the circuit board 60 may be provided with a first connector terminal 66 connected to the microcurrent generator 65, and a second connector terminal 67 connected to the motor driver 64.

The main control unit 61 may transmit or receive mode signal information to or from the mode selector 62 according to a preset program. The main control unit may control the LED generator 63, the motor driver 64, and the microcurrent generator 65 in response to the transceived mode signal information. Furthermore, the main control unit 61 may perceive the charge and discharge state of the battery 50 to provide information about the charge/discharge state to the LED generator 63.

The mode selector 62 may transmit the mode signal information selected by manipulating the button 70 to the main control unit 61. Here, the mode selector 62 may be a portion of the main control unit 61. However, in this embodiment, for the convenience of description, the mode selector and the main control unit will be separately described.

To be more specific, if the button is pressed for a long time (about 1 second) in the state where the cosmetic device 1 is turned off, the mode selector 62 turns on power supplies and simultaneously selects the soft mode. If information about the soft mode signal is transmitted to the main control unit 61, the main control unit 61 may control the LED generator 63, the motor driver 64, and the microcurrent generator 65 in response to the information about the soft mode signal.

Furthermore, if the button 70 is pressed twice in the soft mode, the mode selector 62 selects the power mode. If information about the power mode signal is transmitted to the main control unit 61, the main control unit 61 may control the LED generator 63, the motor driver 64, and the microcurrent generator 65 in response to the information about the power mode signal.

Furthermore, if the button 70 is pressed once in the power mode, the mode selector 62 selects the soft mode again. If information about the soft mode signal is transmitted to the main control unit 61, the main control unit 61 may control the LED generator 63, the motor driver 64, and the microcurrent generator 65 again in response to the information about the soft mode signal.

When the button 70 is not manipulated for a predetermined time, for example, three minutes, the mode selector 62 may transmit manipulation information to the main control unit 61. The main control unit 61 may control to automatically turn off all power supplies in response to the manipulation information.

Moreover, when one desires to turn off the cosmetic device 1 during its use, if the button 70 is pressed for a long time (about 1 second), the mode selector 62 may transmit the manipulation information to the main control unit 61, and the main control unit 61 may control to automatically turn off all power supplies in response to the manipulation information.

The LED generator 63 may give an alarm through the LED lamp 80 to allow a user to visually check what a current mode is, in response to the mode signal information transmitted from the main control unit 61.

To be more specific, the LED generator 63 causes the LED lamp 80 to emit green light if the information about the soft mode signal is transmitted from the main control unit 61, and causes the LED lamp 80 to emit blue light if the information about the power mode signal is transmitted from the main control unit 61, and causes the LED lamp 80 to emit red light if information about the discharge state of the battery 50 is transmitted from the main control unit 61.

The motor driver 64 may drive the vibration motors 40a and 40b in response to the mode signal information transmitted from the main control unit 61.

To be more specific, if the information about the soft mode signal is transmitted from the main control unit 61, the motor driver 64 may drive the vibration motors 40a and 40b at 9,000 RPM to 12,000 RPM that corresponds to about 60% to 80% of the rating. If the information about the power mode signal is transmitted from the main control unit 61, the motor driver 64 may drive the vibration motors 40a and 40b at 15,000 RPM that is the rating level. In this embodiment, if the information about the soft mode signal is transmitted from the main control unit 61, the vibration motors 40a and 40b are driven at 10,500 RPM that is about 70% of the rating level.

The microcurrent generator 65 may generate microcurrent in response to the mode signal information transmitted from the main control unit 61, thus allowing the microcurrent to flow through a microcurrent module into the rotating bodies 20a and 20b. Here, the microcurrent module is not limited to one specific configuration. The microcurrent module is configured such that it causes “positive” current of the battery 50 to flow through any one 30a of the shafts 30a and 30b and the bearing 92 to any one 20a of the rotating bodies 20a and 20b, thus allowing the rotating body 20a to become the “positive” electrode, and it causes “negative” current of the battery 50 to flow through another one 30b of the shafts 30a and 30b and the bearing 92 to another one 20b of the rotating bodies 20a and 20b, thus allowing the rotating body 20b to become the “negative” electrode.

If the rotating body 20a that is the “positive” electrode and the rotating body 20b that is the “negative” electrode simultaneously come into contact with the skin, the microcurrent module forms a closed circuit around the battery 50, and thus the microcurrent is transmitted to the skin. Further, as described above, if the handle 10 is provided with an electrode (not illustrated), when the hand comes into contact with the electrode and at least one of the rotating bodies 20a and 20b comes into contact with the skin, the closed circuit is formed around the battery 50, so that the microcurrent is transmitted to the skin.

To be more specific, if the information about the soft mode signal is transmitted from the main control unit 61, the microcurrent generator 65 generates current of 25 uA to 35 uA to cause the microcurrent to flow to the rotating bodies 20a and 20b. If the information about the power mode signal is transmitted from the main control unit 61, the microcurrent generator 65 generates current of 90 uA to 110 uA to cause the microcurrent to flow to the rotating bodies 20a and 20b. In this embodiment, if the information about the soft mode signal is transmitted from the main control unit 61, the microcurrent generator 65 generates current of 30 uA. If the information about the power mode signal is transmitted from the main control unit 61, the microcurrent generator 65 generates current of 100 uA.

As such, according to this embodiment, the vibration motors 40a and 40b are installed in the shaft members 31 of the shafts 30a and 30b inserted into the rotating bodies 20a and 20b, so that the vibration of the vibration motors 40a and 40b may be directly transmitted to the rotating bodies 20a and 20b, and thereby vibration efficiency may be increased. Moreover, vibration is not transmitted to the hand through the handle 10, thus improving a user's satisfaction as well as a product quality.

Furthermore, according to this embodiment, the vibration motors 40a and 40b are provided not in the handle 10 but in the rotating bodies 20a and 20b, so that a spare space is provided in the handle 10 unlike the related art in which the vibration motor is provided in the handle, and consequently it is possible to make better use of a space and to achieve miniaturization using the spare space.

Furthermore, according to this embodiment, the fixing member 32 is formed to extend from the shaft member 31 of each of the shafts 30a and 30b in the shape of the plate, so that the fixing member 32 may be easily fixed to the handle 10 using the screw 34, thus simplifying a component for fixing the shafts 30a and 30b, and the electric wire is connected to the screw 34, so that microcurrent flows through the shafts 30a and 30b to the rotating bodies, thus realizing the microcurrent massage function.

Although the present disclosure was described with reference to specific embodiments, it is apparent to those skilled in the art that the present disclosure may be changed and modified in various ways without departing from the spirit or scope of the present disclosure, which is described in the following claims. Therefore, it should be interpreted that technical contents related to variations and applications that can be easily derived from embodiments of the present disclosure are included in the present disclosure.

What is claimed is:

1. A cosmetic device, comprising:
a pair of shafts secured to a handle;
a pair of rotating bodies rotatably coupled to the pair of shafts; and
a pair of vibration motors configured to transmit vibration to the pair of rotating bodies,
wherein the vibration motors are installed in the shafts, respectively.

2. The cosmetic device of claim 1, wherein the shafts are independently provided on opposite sides of a partition wall that is formed to have a predetermined size along a center line inside a front end of the handle, so that the shafts are electrically separated from each other.

3. The cosmetic device of claim 1, wherein each of the shafts comprises:
a cylindrical shaft member inserted into a hole of each of the rotating bodies; and
a fixing member extending from the shaft member in a shape of a plate, and fixedly coupled to the handle.

4. The cosmetic device of claim 3, wherein the shaft member comprises an installation space in which each of the vibration motors is inserted and installed.

5. The cosmetic device of claim 1, further comprising:
a battery installed in the handle to supply power; and
a circuit board installed in the handle, and configured to perform a vibration massage function by the vibration motor and a microcurrent massage function by a microcurrent module.

6. The cosmetic device of claim 5, wherein the microcurrent module is configured such that it causes “positive” current of the battery to flow through any one of the shafts and a bearing to any one of the rotating bodies, thus allowing this rotating body to become the “positive” electrode, and it causes “negative” current of the battery to flow through a remaining one of the shafts and the bearing to the remaining one of the rotating bodies, thus allowing this rotating body to become the “negative” electrode.

7. The cosmetic device of claim 6, wherein the microcurrent module forms a closed circuit around the battery if the rotating body that is the “positive” electrode and the rotating body that is the “negative” electrode simultaneously come into contact with the skin, thus transmitting the microcurrent to the skin.

8. A cosmetic device, comprising:
a main control unit;
a mode selector transmitting mode signal information selected by manipulating a button to the main control unit;
an LED generator giving an alarm through an LED lamp to indicate what a current mode is, in response to the mode signal information transmitted from the main control unit;
a motor driver driving a vibration motor installed inn a shaft that may rotate a rotating body, in response to the mode signal information transmitted from the main control unit; and
a microcurrent generator generating microcurrent to cause the microcurrent to flow in the rotating body, in response to the mode signal information transmitted from the main control unit.

9. The cosmetic device of claim 8, wherein the mode selector transmits soft mode signal information selected by pressing the button once, and power mode signal information selected by pressing the button twice to the main control unit.

10. The cosmetic device of claim 9,
wherein the main control unit perceives a charge or discharge state of the battery to provide information about the charge/discharge state to the LED generator, and
wherein the LED generator causes the LED lamp to emit green light if the soft mode signal information is transmitted from the main control unit, and causes the LED lamp to emit blue light if the power mode signal information is transmitted from the main control unit, and causes the LED lamp to emit red light if information about the discharge state of the battery is transmitted from the main control unit.

11. The cosmetic device of claim 9, wherein the motor driver causes the vibration motor to be driven at a level of 60% to 80% of rating, if the soft mode signal information is transmitted from the main control unit, and causes the vibration motor to be driven at a rating level, if the power mode signal information is transmitted from the main control unit.

12. The cosmetic device of claim 9, wherein the microcurrent generator generates current of 25 uA to 35 uA if the soft mode signal information is transmitted from the main control unit, and generates current of 90 uA to 110 uA if the power mode signal information is transmitted from the main control unit.
